(19) United States
(12) Patent Application Publication
(10) Pub. No.: US 2022/0296831 A1
(43) Pub. Date: Sep. 22, 2022

(54) BREATHING CONTROL DEVICE

(71) Applicant: T & G Family Inventions, LLC, Hobe Sound, FL (US)

(72) Inventors: Larry A. Thomas, Hobe Sound, FL (US); Jeannie M. Gipson, Hobe Sound, FL (US)

(21) Appl. No.: 17/655,087

(22) Filed: Mar. 16, 2022

Related U.S. Application Data
(60) Provisional application No. 63/162,077, filed on Mar. 17, 2021.

(51) Int. Cl. A61M 16/00; A61M 16/20

(52) U.S. Cl. CPC A61M 16/0045; A61M 16/208

(57) ABSTRACT

A breathing control device formed from a regulator having a stem opening and aperture opening. The regulator having a mouthpiece for inhaling and exhaling into a diverter bag coupled to the regulator. The stem opening and aperture opening available to control the amount of ambient air drawn into regulator to regulate the ratio of carbon dioxide to oxygen level drawn from the diverter bag to assist in treating panic and anxiety attacks associated with hyperventilation and migraines. The diverter bag retaining the exhaled breath expelled by the individual for reuse, the aperture opening can be capped to limit all ambient air through the stem opening. The diverter bag becomes a visual focus as exhaled breathing causes the bag to unfold with thought provoking indicia.

### PRIORITY CLAIM

In accordance with 37 C.F.R. 1.76, a claim of priority is included in an Application Data Sheet filed concurrently herewith. Accordingly, the present invention claims priority to U.S. Provisional Patent Application No. 63/162,077, entitled “ANXIETY AND PANIC BLOCKER”, filed Mar. 17, 2021. The contents of which the above referenced application is incorporated herein by reference.

### FIELD OF THE INVENTION

This invention is directed to the field of mental health care; and, in particular, to a breathing control device to relieve anxiety attacks, panic attacks, and migraines by controlling the balance of air and carbon dioxide.

### BACKGROUND OF THE INVENTION

Anxiety is the mind and body's reaction to stressful, dangerous, or unfamiliar situations is generally characterized as uncontrollable worrying about events and activities leaving a feeling of apprehension. The individual may have unexplained thoughts of potential negative outcomes or impending doom. While it is commonplace for an individual to feel anxiety at stressful times in their lives, anxiety disorders may be a result of genetics, personality, family traits or certain life events. For those suffering from excess anxiety, anxiety disorder and/or panic attacks, the feeling is far from normal and is often debilitating. Left unchecked, anxiety and panic attacks can significantly diminish an individual's quality of life.

Anxiety and panic attacks is a side effect from hyperventilating. Hyperventilating may go unnoticed when it first starts, and too late when detected. Signs of an attack include: 1) individual's thoughts become negative or overwhelming, 2) individual begins to worry, 2) individual's heartbeat rate increases, and 4) individual has an indescribable feeling throughout their body—sometimes with sweating. Once the individual is in a state of hyperventilating, the depletion of carbon dioxide has started and the required balance of air and carbon dioxide has changed. For purposes of this specification inhaled air is defined as 21% oxygen, 78% nitrogen, argon 0.93%, carbon dioxide 0.04%, and other gases. Exhaled air is 78% nitrogen, 16% oxygen, 4% carbon dioxide and other gases. Once the anxiety or panic attack begins, hyperventilation causes the exhaled carbon dioxide to increase. As a result the bloods concentration is reduced and the bicarbonate/carbonic acid alters the pH in the blood.

Anxiety and panic attacks is such a prevalent problem that entire fields have been developed to address treatments, many of which require the assistance of mental health experts or other licensed professionals. Such treatments include cognitive behavioral therapy (CBT) to develop coping skills. Dialectical behavioral therapy (DBT) is directed to a practice of examining multiple or often contradictory ideas and enabling individuals to recognize and attempt to understand thoughts as they occur. Group Therapy helps to normalize an individual's experience by relating to others. Hypnosis is used to help people achieve a relaxed state through breathing, guided imagery, or muscle-relaxing techniques.

More severe anxiety and panic attack cases may necessitate prescription medications, such as antidepressants named on how they work in the brain, selective serotonin reuptake inhibitors (SSRIs), and serotonin-norepinephrine reuptake inhibitors (SNRIs). Serotonin is a chemical that acts as a neurotransmitter, carrying signals along and between nerves; and it also plays a role in mood regulation, which is helpful for someone with anxiety and panic attacks. Benzodiazepines work on the GABA (gamma-aminobutyric acid) neurotransmitter, which plays numerous roles related to sleep, relaxation, anxiety, mood changes, and memory. Beta Blockers block the binding of epinephrine (adrenaline) and norepinephrine to nerve receptors. Other medications include tricyclic antidepressant (imipramine); a monoamine oxidase inhibitor, or MAO inhibitor; an anxiolytic (buspirone).

Professional assistance can be prohibitively expensive and not always productive. For this reason, many consumers have turned to self-help methods to treat anxiety and panic attacks. For instance, exercise can cause the body to release endorphins, which are hormones that can improve your mood. Diet corrections can help to treat nutrient deficiencies and different manifestations of anxiety and panic attacks. Meditation is helpful to focus thoughts in one specific direction and redirect them when they begin to wander. Relaxation techniques to help people relax, including guided imagery and breathing exercises for slowing the heart rate and focusing thoughts, are also common techniques.

What is lacking in art is a device that can help treat anxiety and panic attacks by controlling one's breathing and balancing the carbon dioxide and oxygen.

### SUMMARY OF THE INVENTION

Disclosed is a portable breathing control device that provides for reestablishing a proper oxygen and carbon dioxide level in an individual that is hyperventilating. The device is includes a regulator having a stem opening and aperture extending outwardly therefrom. The regulator having a mouthpiece for inhaling and exhaling into a diverter bag coupled to the regulator to control the amount of ambient air drawn into regulator. The result is to regulate the ratio of carbon dioxide drawn from the diverter bag with ambient air to assist in treating panic and anxiety attacks associated with hyperventilation and migraines. The diverter bag retaining the exhaled breath expelled by the individual for reuse. The opening in the regulator can be capped to restrict most ambient air through the stem opening.

The instant device limits the amount of ambient air reused, and the diverter bag becomes a visual representation of the individual's breathing, which causes the bag to unfold with thought provoking indicia displayed during the breathing process. The regulator allow ambient air into the passageway to be admixed with the individual's stored breath, with a predetermined aperture sized for conventional or severe attacks. The combination of the controlled breathing, partial use of carbon dioxide, and visual stimulation provides effective anxiety and panic attack therapy.

An objective of the invention is to provide a portable device for treating anxiety and panic attacks, or migraines, by control of the ration of air to carbon dioxide entering the lungs.

Another objective of the invention is to provide a mouth piece having a valve that allows an admixture of carbon dioxide and air entering the lungs.

Still another objective of the invention is to provide a regulator cap for changing the admixture level of ambient air to carbon dioxide entering the lungs.

Still another objective of the invention is to a movable bag to help focus an individual's thoughts while relieving anxiety and panic attacks.

Yet another objective of the invention is to provide a device that having a foldable bag and a compact mouthpiece that can be stores in small places such as a purse, pants or shirt pocket.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE DRAWINGS

FIG. 1 is a pictorial front view of the breathing control device;

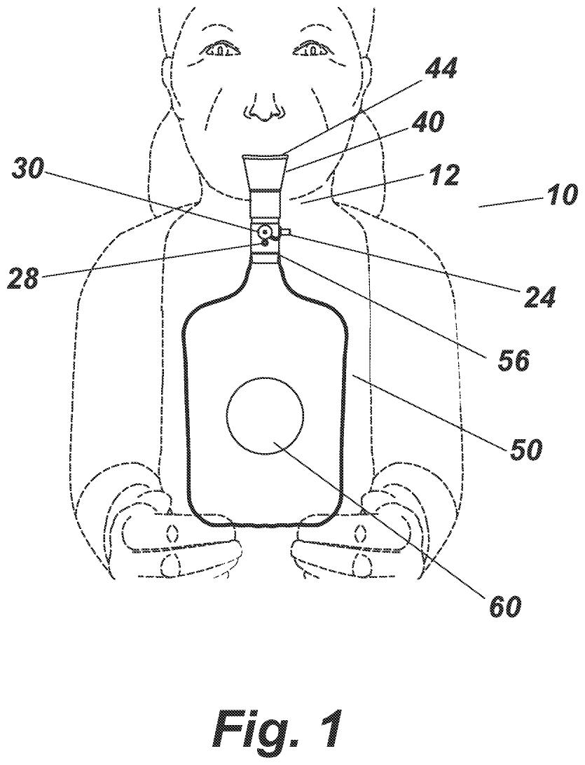

FIG. 2 is a perspective view of the mouthpiece;

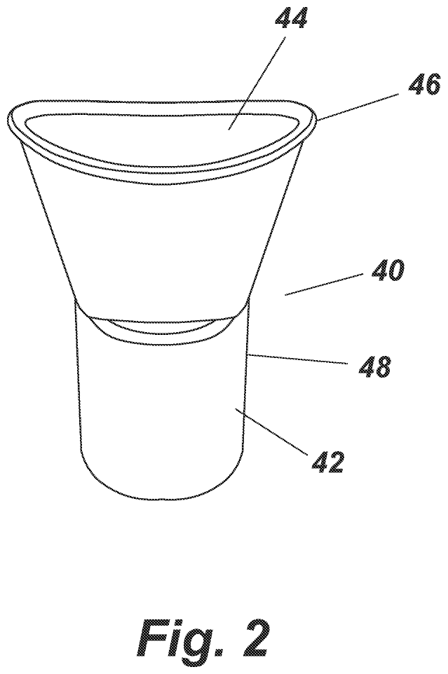

FIG. 3 is a perspective view of the regulator;

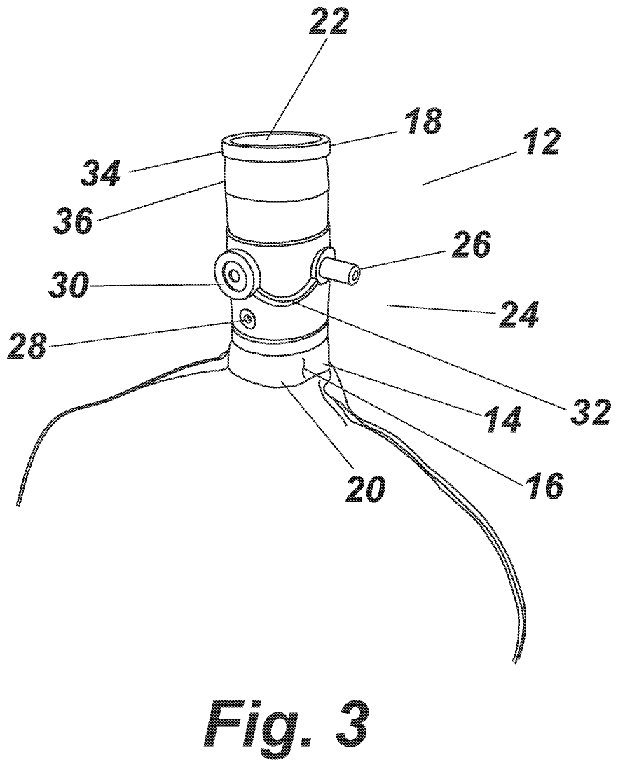

FIG. 4 is a perspective view of the diverter bag.

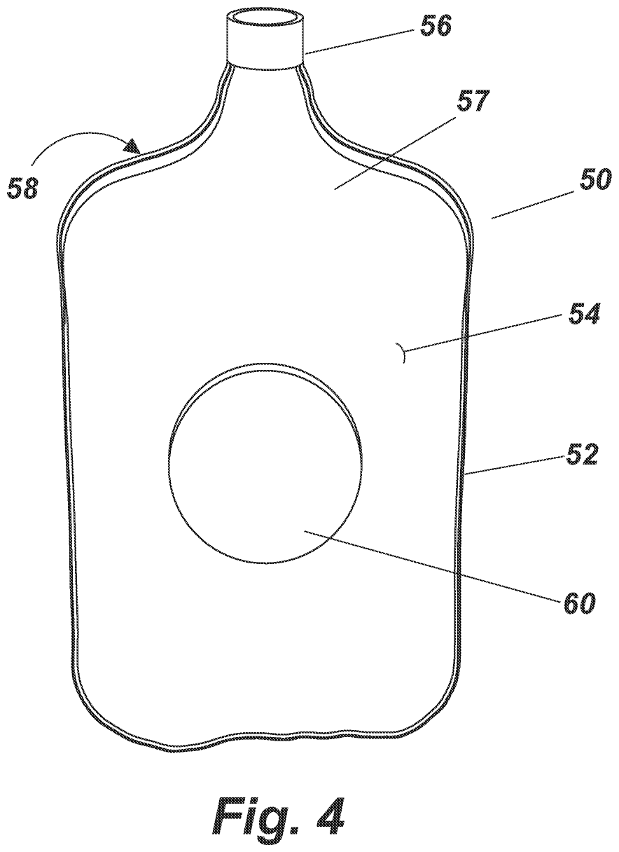

### DETAILED DESCRIPTION OF THE PREFERRED EMBODIMENT

A detailed embodiment of the instant invention is disclosed herein; however, it is to be understood that the disclosed embodiment is merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Referring now to the Figures, illustrated is a breathing control device comprising a regulator 12 formed from a tube 12 having a continuous side wall 16 with a leading edge 18 and a trailing edge 20. The side wall 16 forming an inner passageway 22 between the leading edge 18 and the trailing edge 20. A stem 24 extends outwardly from the side wall 16. The stem 24 having a stem opening 26 of a predetermined size being 1/16 inch that is fluidly communicating with the passageway 22. The stem opening 26 provides an unrestricted draw of ambient air, limited by the size of the stem opening, for drawing ambient air into the passageway 22. An aperture 28 is formed in the side wall 16 providing a ⅛ inch opening to the passageway 22, for drawing ambient air into the passageway. Unlike the stem, the aperture can restrict the flow of air by use of a cap 30. The cap 30 is used in the event of a major anxiety or panic attack, or severe migraine headache where ambient air being drawn into the passageway 22 is limited to the stem opening 26.

For ease of access, the cap 30 is attached to the stem 24 by a strap 32. The strap 32 maintains the cap 30 in close proximity to the aperture 28 for sealing the aperture during the major anxiety or panic attack, or severe migraine headache. For less strenuous situations, the aperture 28 is left open for drawing in a larger volume of ambient air than possible with the stem opening 26.

In a preferred embodiment the leading edge 18 of the regulator 12 includes a sealing lip 34 having a diameter and thickness greater than the neck section 36 of the regulator 12. The sidewall 16 is preferable of a thickness greater than the neck section 36, providing additional support to the stem 24 and aperture 28. The regulator 12 preferred construction is transparent plastic.

A mouthpiece 40 having a tubular shaped outlet 42 is securable to the leading edge 18 of the regulator 12. An oval shaped inlet 44 is formed along an outer edge 46 which is constructed and arranged to adapt to the shape of an individual's mouth to allow inhaling and exhaling through the inlet 44. The outlet is sized to slide over the sealing lip 34 seal the mouthpiece 40 to the regulator 12. In the preferred embodiment, the mouthpiece sleeve 48 fits over the regulator neck 36. The mouthpiece 40 preferred construction is transparent plastic.

A diverter bag 50 having a sealed edge 52 forming a volume chamber 54 within the diverter bag 50 having a circular shaped inlet 56 securable to the trailing edge 20 of the regulator 12. In one embodiment the shaped inlet 54 can be secured to side wall 16 by an adhesive or adhesive tape sealing the shaped inlet 56 to the regulator 12. The diverter bag 50 is preferable formed of an upper flexible plastic sheet 57 attached to a lower flexible plastic sheet 58 by a weldment along an adjoined edge 52 forming the flexible volume chamber 54 therebetween.

In operation, step 1—the cap 30 is removed from the aperture 28; step 2—an individual inhales ambient air for 5 seconds and holds that breath for 5 seconds; step 3—the individual exhales into the mouthpiece 40 for 5 seconds; step 4—the individual inhales for 5 seconds drawing ambient air through said stem opening 26 and aperture 28 to combine with the air held in the volume chamber 54, and then exhales for 5 seconds refilling the volume chamber 54 with excess air exhausted through the stem opening 26 and aperture, step four is repeated four times. The stem opening 26 and aperture are calculated to allow a predetermined amount of ambient air into the regulator to allow a rebalance of the proper oxygen to carbon dioxide ratio. In the event of a major anxiety or panic attack, or severe migraine headache, the cap 30 is placed over the aperture 38 and the aforementioned steps performed.

The diverter bag 50 receives a portion of the individual's exhaled breath which contains carbon dioxide, nitrogen and oxygen. The diverter bag retains this during a breath. An anxiety and panic attack is typically manifested by hyperventilating wherein an individual may breathe too fast which causes the body to lose carbon dioxide beyond an acceptable level. While carbon dioxide is a byproduct of exhaling, a minimal amount remains necessary for the body to maintain a proper bloodstream pH balance. Should the individual loose too much carbon dioxide, especially during hyperventilating, the body can start to malfunction. When an individual exhales in the chamber, a flow of the individual's breath containing carbon dioxide is directed into the diverter bag through the regulator, the breath causing movement of the bag, and excess air is expelled. As the diverter bag expands, the movement thereof and the inclusion of indicia 60 on an outer surface 57 of the diverter bag 50 has been found to help an individual focus their thoughts on the breathing to help lessen the attack. The indicia may be colors or designs particularly chosen to help an individual focus their thoughts.

The breathing control device 10 can be stored in a compact position wherein the diverter bag 50 is folded into a size that can equal the size of the regulator and mouthpiece. In addition, the mouthpiece can be removed from the regulator for further compactness allowing the device 10 to carried with an individual at all times so as to immediately address an attack. In one embodiment the device is stored in small convenient sized storage pouch.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. The stated time span of 5 seconds is considered the preferred time, although other times are acceptable and considered within the scope of this invention, as times may vary based upon the lung capacity of the individual.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A breathing control device comprising:
a regulator formed from a tube having a continuous side wall with a leading edge and a trailing edge, said side wall forming an inner passageway between said leading edge and said trailing edge;
a stem extending outwardly from said side wall, said stem having an opening of a predetermined size that is fluidly communicating with said passageway, wherein said opening is unrestricted to allow an individual to draw air a controlled amount of air into said passageway;
an aperture formed in said side wall and fluidly communicating with said passageway;
a cap securable to said aperture, said cap adapted to restrict air flow into said passageway; and
a diverter bag having an inlet securable to said trailing edge of said regulator, said diverter bag having a volume chamber.

2. The breathing control device according to claim 1 including a mouthpiece having a tubular shaped outlet securable to said leading edge of said regulator, and an oval shaped inlet, wherein said mouthpiece is constructed and arranged to adapt to the shape of an individual's mouth to allow inhaling and exhaling through said inlet.

3. The breathing control device according to claim 1 wherein said stem is positioned perpendicular to said aperture.

4. The breathing control device according to claim 1 wherein said diverter bag is flexible, wherein an individual having anxiety issues exhales into said diverter bag causing movement thereof.

5. The breathing control device according to claim 4 wherein said diverter bag includes indicia constructed and arranged to help focus an individual's attention while said diverter bag expands and contracts.

6. The breathing control device according to claim 4 wherein said diverter bag is biased in a folded position for compact storage.

7. The breathing control device according to claim 1 wherein diverter bag provides a predetermined backpressure to limit the amount of exhaled air retained while in an expanded position.

8. The breathing control device according to claim 1 wherein said stem opening is 1/16 inch.

9. The breathing control device according to claim 1 wherein said aperture is ⅛ inch.

10. The breathing control device according to claim 1 wherein said cap is attached to said stem by a strap wherein said strap maintains said cap in close proximity to said aperture.

11. The breathing control device according to claim 2 wherein said first end of said regulator includes a lip for sealing to an inner surface of said mouthpiece.

12. A breathing control device comprising:
a regulator formed from a tube having a continuous side wall with a leading edge and a trailing edge, said side wall forming an inner passageway between said leading edge and said trailing edge;
a stem extending outwardly from said side wall, said stem having an opening of a predetermined size that is fluidly communicating with said passageway, wherein said opening is unrestricted to allow an individual to draw air a controlled amount of air into said passageway;
an aperture formed in said side wall and fluidly communicating with said passageway;
a cap securable to said aperture, said cap adapted to restrict air flow into said passageway; and
a diverter bag having an inlet securable to said trailing edge of said regulator, said diverter bag having a flexible volume chamber; and
a mouthpiece having a tubular shaped outlet securable to said leading edge of said regulator, and an oval shaped inlet, wherein said mouthpiece is constructed and arranged to adapt to the shape of an individual's mouth to allow inhaling and exhaling through said inlet
wherein an individual exhales through said mouthpiece filling said volume chamber for a first period of time with excess air released through said opening and said aperture, wherein the individual inhales through said mouthpiece drawing exhaled air from said volume chamber together with a predetermined amount of ambient air from said opening and said aperture, whereby said exhaling and inhaling is repeated a plurality of times.

13. The breathing control device according to claim 12 wherein said stem is positioned perpendicular to said aperture.

14. The breathing control device according to claim 1 wherein said diverter bag is flexible, wherein an individual having anxiety issues exhales into said diverter bag causing movement thereof.

15. The breathing control device according to claim 12 wherein said diverter bag is biased in a folded position for compact storage.

16. The breathing control device according to claim 12 wherein said stem opening is 1/16 inch.

17. The breathing control device according to claim 12 wherein said aperture is ⅛ inch.

18. The breathing control device according to claim 12 wherein said cap is attached to said stem by a strap wherein said strap maintains said cap in close proximity to said aperture.

19. The breathing control device according to claim 12 wherein said predetermined time is 5 seconds.
